(19) United States
(12) Patent Application Publication
(10) Pub. No.: US 2017/0265934 A1
(43) Pub. Date: Sep. 21, 2017

(54) ELECTRO-CAUTERIZING BI-POLAR SURGICAL FORCEPS

(71) Applicants: Tyrone Secord, Rochester Hills, MI (US); Chaim Colen, Pompano Beach, FL (US); Michael Anderson, Grosse Pointe Farms, MI (US)

(72) Inventors: Tyrone Secord, Rochester Hills, MI (US); Chaim Colen, Pompano Beach, FL (US); Michael Anderson, Grosse Pointe Farms, MI (US)

(21) Appl. No.: 15/458,598

(22) Filed: Mar. 14, 2017

Related U.S. Application Data
(60) Provisional application No. 62/309,906, filed on Mar. 17, 2016.

(51) Int. Cl. A61B 18/14; A61B 18/12

(52) U.S. Cl. CPC A61B 18/1445; A61B 18/1206; A61B 2018/1462; A61B 2018/145; A61B 2018/126

(57) ABSTRACT

A bi-polar forceps for providing electro-cauterization of tissue and blood vessels including first and second elongated beams extending from a rearmost interconnected end to forward most separated probe ends. The beams each include rearmost base portions, intermediate angled portions and forward most extending portions such that the probe ends are established in a non-linear arrangement relative to the rearmost portions. First and second pins extend from the rear end and, upon activating with the probe ends placed in communication with a tissue or vein location to be cauterized, electrically communicate with the forward probe ends.

### CROSS REFERENCE TO RELATED APPLICATIONS

The present invention claims the priority of U.S. Ser. No. 62/309,906 filed Mar. 17, 2016, the contents of which are incorporated herein in ites entirety.

### FIELD OF THE INVENTION

The present invention discloses an electro-cauterizing and bi-polar surgical forceps utilizing a pair of elongated metal and electrically conductive substrates with electrical contact pins at first input ends and conducting tips and second forward ends. The body design of the forceps is unique with both plastic over-mold locations along the outer opposite gripping surfaces, as well as opposing and inner facing interlocking features established between the pincer halves in order to avoid scissoring of the beams past one another when pincered. This plastic over-molded detail along with a stepped intermediate detail of each pincer beam, provides both enhanced gripping and better visibility during in-situ use.

### BACKGROUND OF THE INVENTION

The concept of an electrocautery bipolar forceps for cauterizing tissue and blood vessels is well known in the art. Such electrosurgical tools are typically powered by a type of electrosurgical generator (see by non-limiting example such as shown in FIG. 1 of the electrosurgical tool of Thorne et al., US 2009/0138013). In operation, the metallic and electrically conductive beams are heated by the electrical current passed from the electrosurgical generator (i.e. typically by an arrangement of resistors integrated into the supporting circuitry of the probe and which result in the generation of heat passed by conductivity from the probe ends of the joined (closed circuit) beams into the tissue or blood vessel in order to seal off or coagulate bleeding at the location.

Additional references of note also include the non-stick electrosurgical forceps of Thorne, U.S. Pat. No. 6,293,946, the instrument of Patton US 2007/0276363, and the disposable bi-polar instrument of Williams U.S. Pat. No. 4,686,980. Also referenced are the non-stick bipolar forceps of Hanlon US 2008/0200914 and the plasma bipolar forceps of Evans US 2013/0066317.

### SUMMARY OF THE PRESENT INVENTION

The present invention discloses a bi-polar forceps for providing electro-cauterization of tissue and blood vessels including first and second elongated beams extending from a rearmost interconnected end to forward most separated probe ends. The beams each include rearmost base portions, intermediate angled portions and forward most extending portions such that the probe ends are established in a non-linear arrangement relative to the rearmost portions. First and second pins extend from the rear end and, upon activating with the probe ends placed in communication with a tissue or vein location to be cauterized, electrically communicate with the forward probe ends.

Additional features include a plastic material over-molding a substantial length of each of the beams short of the probe ends. A second over-molded plastic plug can be provided which encapsulates the rear ends of the beams and from which extends the pins.

The forceps as described in claim 2, further comprising pluralities of user gripping embossments arranged upon opposite facing surfaces of at least one of said rearmost or intermediate angled portions of each of said beams. The forceps further exhibits interlocking features extending from inner opposing surfaces of the beams for preventing scissoring of the beams during pincering together of the forward most probe ends.

An additional variant incorporates into each beam an inner elongated wire form, having any cross sectional shape, and which is over molded with the plastic material (such as by a two stage injection molding process or a sonic welding process for joining two previously molded halves supporting the wire form therebetween). The wire forms each exhibit a flattened shape at the forward probe ends where the two sides contact, in addition to round electrical contacts on the opposing ends, at a smaller diameter, and in order to interface with the wire connector that provides electrical current. As with the previous embodiment, both sides have the plastic over-molded housing on the rear end which can be provided as an end cap attachment fitting over the projecting pin ends of the wireforms.

A yet further related variant reconfigures the wire forms as thin metal plated or etched surfaces (gold, silver or copper) which are configured within a selected mating half of individual pairs of mating halves configuring each plastic beam, the metal or etched plating extending the length of the beams. Each beam integrated metal plating expands in dimension to define forward probe pincer ends, these providing the pinch feature that makes tissue contact and provides electrical current. The opposing rear ends of each etched plate or layer again providing the electrical contact to interface with the wire connector.

As will be further described, and in one version, the metal plating is again applied to only one of the two halves, with the second being a separate component that is heat or sonic welded to the metal plated half. Alternatively, the second of each joined pair of halves can be over-molded to create the assembly for each side, such after the metal plating process. As with the previous disclosed variant, the two sides are assembled together with the plastic over-molded housing secured to the opposite rear ends, with the electrical contacts extending beyond the housing.

A related metal plating process for creating the metal plated electro cauterizing bipolar forceps employs current technology and utilizes a plastic composite with a metal additive. The desired metal plated surfaces or configurations may be initially laser etched to remove plastic material (minimal depth) and to expose the metal additive, which is then plated with the desired metal to provide the electrical conduit through the part.

### BRIEF DESCRIPTION OF THE DRAWINGS

Reference will now be made to the attached drawings, when read in combination with the following detailed description, wherein like reference numerals refer to like parts throughout the several views, and in which:

FIG. 1 is a right side plan view of the electro-cauterizing bi-polar surgical forceps according to one non-limiting embodiment of the present invention;

FIG. 2 is a bottom plan view of the present invention;

FIG. 3 is a left side plan view of the present invention;

FIG. 4 is a top plan view of the present invention;

FIG. 5 is a front plan view of the present invention;

FIG. 6 is a rear plan view of the present invention;

FIG. 7 is a first topside looking perspective view of the present invention;

FIG. 8 is a second rotated and underside looking perspective of the present invention;

FIG. 9 is a perspective illustration similar to that shown in FIG. 7 and depicting a further variant of bipolar surgical forceps integrating a wire form within each beam;

FIG. 10 is a top elongated view of the embodiment of FIG. 9;

FIG. 11A is a length extending cutaway view taken along line 11A-11A of FIG. 10 of a given beam and depicting the inner wire form;

FIG. 11B is a crosswise extending cutaway view taken along line 11B-11B of FIG. 10 at an intermediate location and illustrating wire form embedded within the polymer over molding;

FIG. 12 is an exploded view of the bi-polar surgical forceps of FIG. 9 with the elongated and configured wire forms separated from the over molded plastic bodies;

FIG. 13 is a rotated perspective view of the surgical forceps of FIG. 9 with an outer facing beam depicted in partial lengthwise cutaway in order to reveal the interior extending wire form;

FIG. 14 is a top elongated view of a yet further variant of bi-polar surgical forceps and illustrating a pair of metal plated or etched conduits which extend the length of each of the beams;

FIG. 15 is a front end view of the surgical forceps of FIG. 14 and again illustrating the offset nature of the extending beams for providing enhanced and non-obstructed viewing by the surgeon during in situ surgical application;

FIG. 16 is a ninety degree rotated angle of the forceps taken along cutaway line 16-16 in FIG. 14, and illustrating the metal plated interior of the indicated beam, such omitting the need for metal stamping or wire forms;

FIG. 17 is a crosswise cutaway taken along line 17-17 of FIG. 14 and depicting a probe end of a selected beam which includes an expanded inner facing contact portion associated with each conductive metal plating;

FIG. 18 is a further crosswise cutaway taken along line 18-18 of FIG. 14 and depicting the thin inner profile of the metal plating extending rearwardly from the forward probe tip ends;

FIG. 19 is a yet further cutaway taken along line 19-19 of FIG. 14 and depicting a rear contact pin end extending past the rear plastic over mold housing, the pin including metal plated inside facing half bonded to a plasticized outer half;

FIG. 20 is a rotated perspective view of the surgical forceps of FIG. 14 with an outer facing beam depicted in partial lengthwise cutaway in order to reveal the interior metal plated portion which is laser etched or otherwise, integrated into an over molded or sonically joined beam;

FIG. 21 is an enlarged partial rear end perspective of FIG. 20, with the outside half facing plastic material removed for better illustrating the inside metal plated surface of the selected contact pin;

FIG. 22 is an enlarged partial front end perspective of FIG. 20 which likewise depicts the outside plastic material removed for better illustrating the inside metal plated probe/tip surfaces;

FIG. 23 is an exploded view the bi-polar surgical forceps of FIG. 14 with the elongated and configured metal conductive platings separated from the over molded. plastic bodies;

FIG. 24 is a cutaway of a probe tip end similar to that shown in FIG. 17 and illustrating an alternate variant for accomplishing secure attachment of a metal layer at each inwardly facing probe end; and

FIG. 25 is a partial forward end perspective of a pair of inwardly facing probe ends as shown in FIG. 24.

### DETAILED DESCRIPTION OF THE PREFERRED EMBODIMENTS

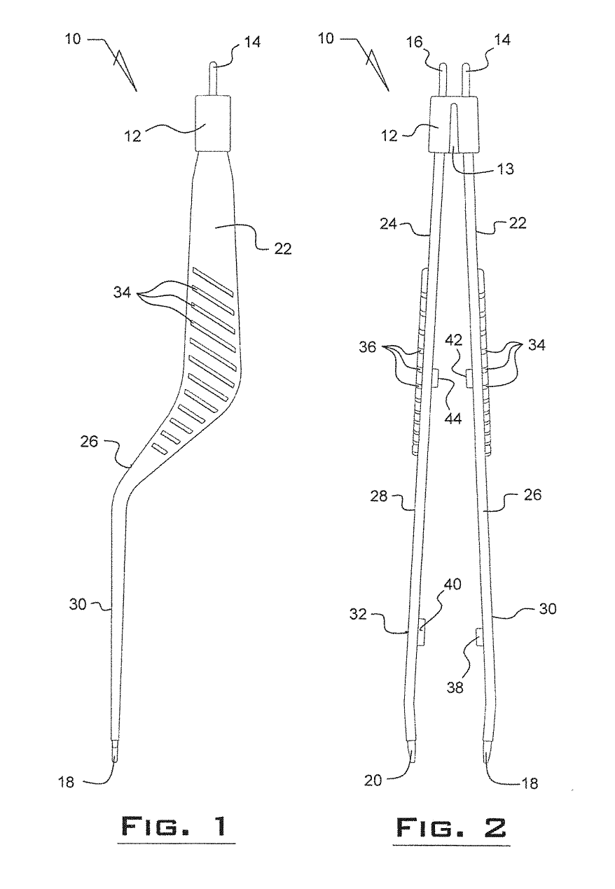

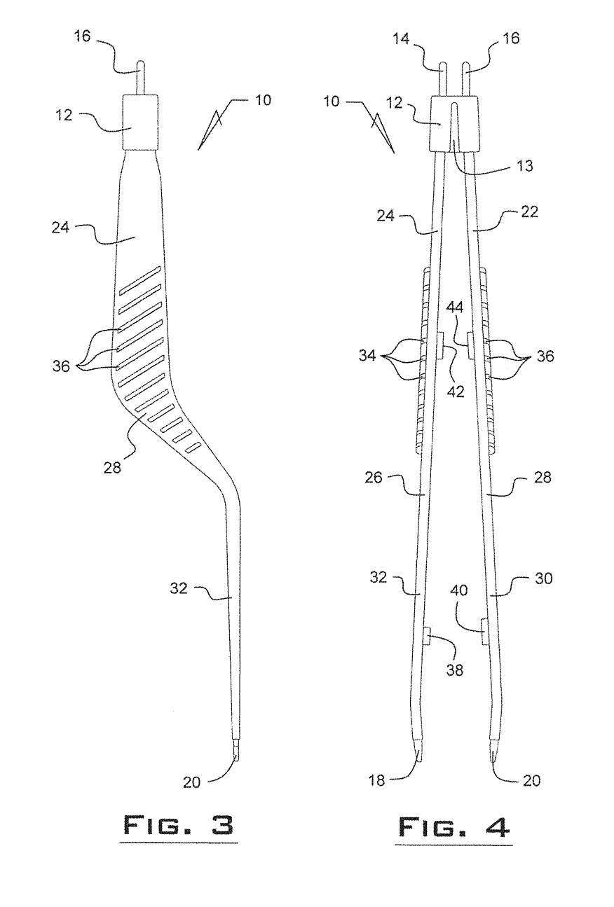

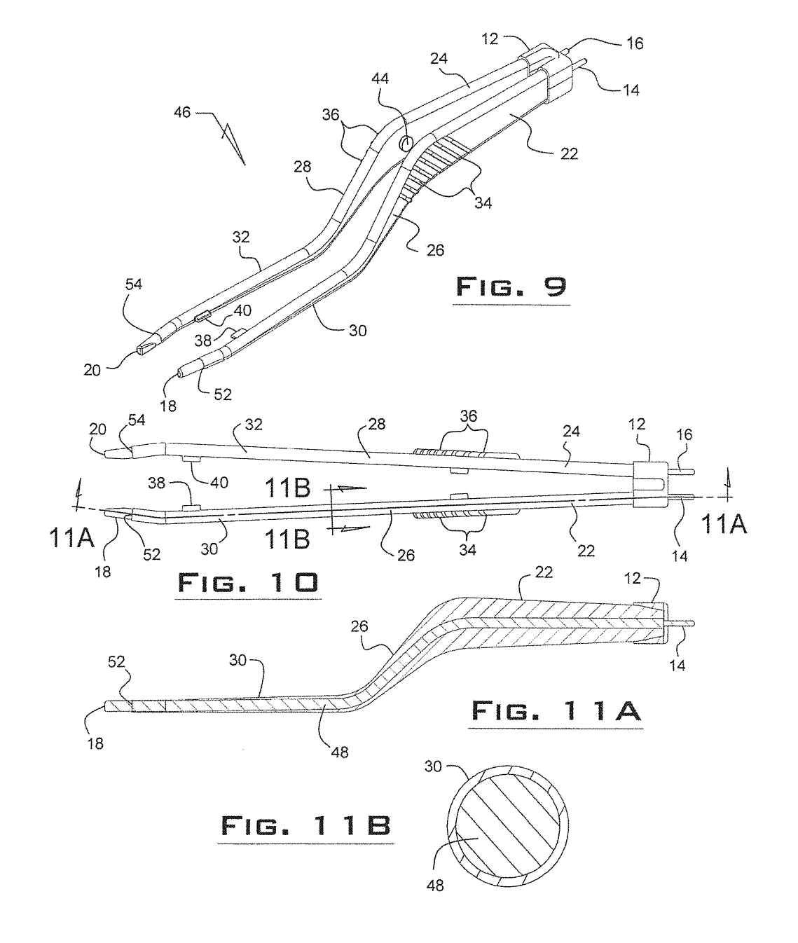

Referring now to FIGS. 1-8, along with further reference to each of FIGS. 9-13 and FIGS. 14-23, the present invention provides a collection of plan and perspective views of related variants of an Electro-Cauterizing Bi-Polar Surgical Forceps according to non-limiting embodiments of the present invention. As will be described in further detail, the configuration of the bi-polar surgical forceps as shown can he modified without departing from the scope of the invention.

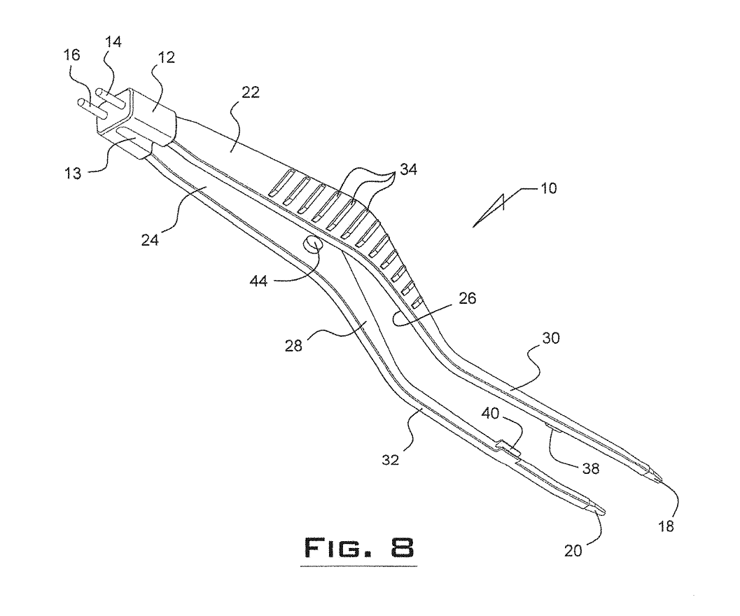

A first variant of the forceps, generally depicted by body 10 in FIGS. 1-8, and as best shown in the rotated perspective views of FIGS. 7-8, includes a pair of elongated beams or pincers which exhibit interior metallic and electrically conductive substrates and which extends from a rear plug end 12 (this also including an over molded or separately attachable plug) from which extends electrical contact pins 14 and 16 to which a cord, not shown, associated with the electrical supply is communicated). The plug 12 can include a secondary plastic which is over-molded about the pre-positioned and extending rear ends of the beams, the plug exhibiting a central depth extending notch or recess 13 terminating short of its rear end face from which extends the pins 14/16, this recessed configuration separating a pair of receiving pockets (see as shown in exploded views of FIGS. 12 and 23) defined in the plug 12 for receiving the rear ends of the beams, and so that the pin 14/16 ends extend through the base of the plug 12. Upon being mounted to the plug 12, the extending beams are provided with the necessary degree of flex or resiliency. Without limitation, the plug 12 can be reconfigured to other shapes such as including a cylindrical or sleeve shape.

The pins 14 and 16 respectively communicate with the metallic interior substrate of the beams, these being better illustrated in the related variants of FIGS. 9-13 and 14-13, and each of which extends the length of the associated beam or pincer (and within a plastic insulating and over-molded gripping material as will be further discussed). The individual metal substrates each conclude at forward most probe ends, see further at 18 and 20, and so that, upon pincering the beams together so that probe ends 18 and 20 contact one another, a circuit is closed and cauterizing heat his allowed to flow through the bi-polar forceps, past the metal tips 18/20, and to the surrounding tissue or blood vessel region in order to provide the desired cauterizing.

The majority of the extending length of each of the beams (from the rear plug end 12 to the forward-most probe ends 18 and 20) is coated by the insulating plastic material, the pincer joined beams each further including rear-most portions 22 and 24, intermediate and downwardly angled (from an upper looking perspective) interconnecting portions 26 and 28, and concluding with forward most extending and interconnecting portions 30 and 32 (these generally however not necessarily co-linear with the rearward most portions 22 and 24) and so that the forward most portions and probe ends are established in a non-linear arrangement relative to said rearmost portions (an advantage of this non-liner shaping providing unobstructed viewing to the surgeon during in situ operation of the device). As further shown, the body design of the forceps is unique with both plastic over-mold locations, see at 34 and 36 respectively, along the outer opposite gripping surfaces of the pincer beams.

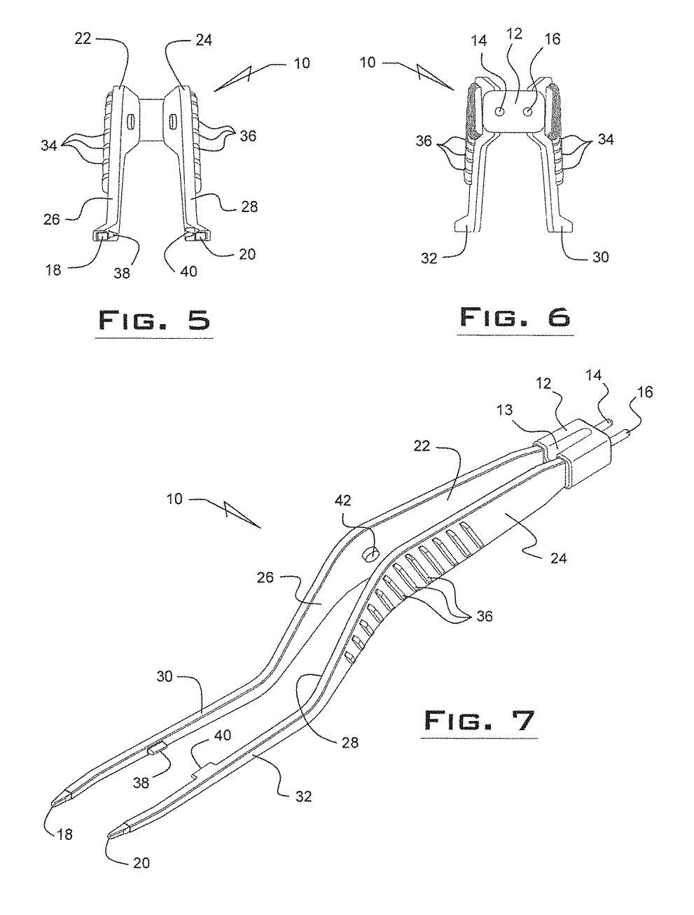

A pair of opposing and inner facing interlocking features are established between the pincer halves (see in particular profiles 38 and 40 associated with the inner facing surfaces of the forward-most beam portions 30 and 32). As best shown in the perspective views of FIGS. 7 and 8 viewed in combination with the front end view of FIG. 5, the first interlocking profile 38 exhibits a generally arrow or blade tip (male) profile which seats within a channel or recess (female) associated with the second opposing and interlocking profile 40 (best shown in FIG. 5 and defined by a pair of projecting outer walls which define the female profile for seating the male profile 38). In this fashion, and upon closing the beams toward one another so that the conductive probe ends 18/20 inter-contact in a circuit closing fashion, the interlocking profiles 38/40 inter-seat with one another in order to avoid scissoring of the beams past one another when pincered together.

A second additional or single alternating interlocking pair of inner facing profiles, see at 42 and 44, can be integrated into further aligning and linearly spaced inwardly opposing surface locations of the inner beam faces of the beams or pincers. In one non-limiting arrangement, such a secondary pair of aligning locations may be provided between the main body locations 22 and 24 of the pincer halves (closer to the interconnected input end 12), and can be in the form of inner opposing seat and projection portions (again at 42/44) without limit as to particular configuration and in order to provide additional spacing and aligning support to the tool inside surfaces as the beams are pincered together into a close circuit contact configuration.

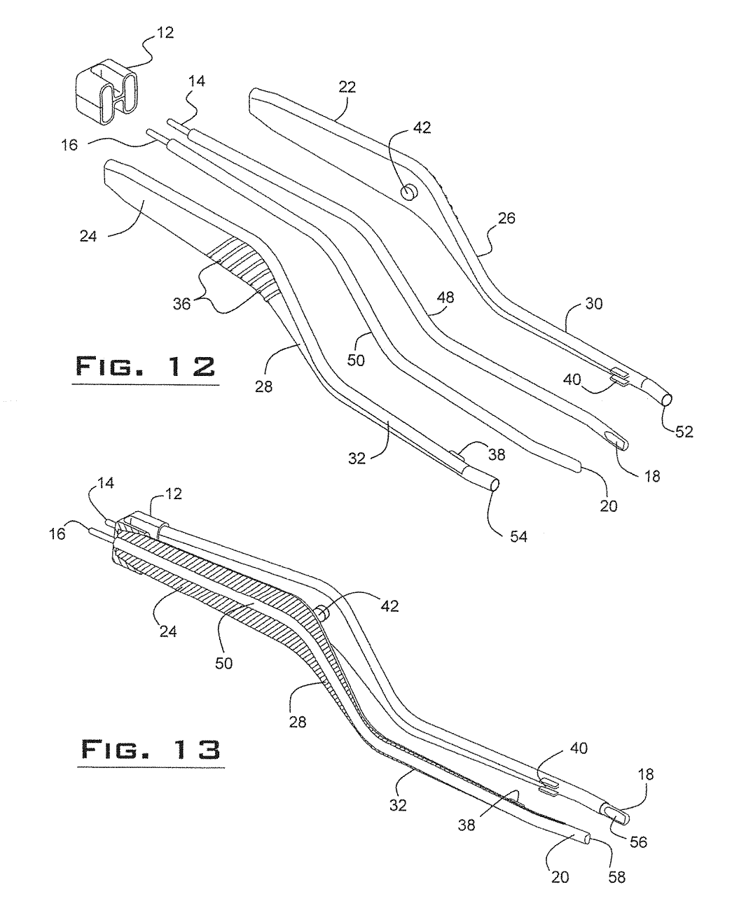

The side profile views of the device as further depicted, in particular the intermediate stepped configuration established by portions 26 and 28, assist in both gripping as well as providing enhanced visibility during in situ surgical use, in particular when gripped by the surgeon in such a position the forward most portions 30/32 and concluding conducting probes 18/20, as substantially shown in FIG. 11B, and which are displaced upwardly relative to the hand gripping rear most 22/24 and intermediate stepped 26/28 portions. In this fashion, the line of sight between the surgeon and the probe tips of the bi-polar forceps is maintained and not obscured by the hand gripping and manipulating the beams.

The plastic over-molded detail shown in the various illustrations can also include the linear profile of the spaced apart gripping portions 34 and 36 being substituted by other recessed or embossed patterns for enhancing the gripping action of the user (particularly when wearing surgical gloves), this again during in-situ use.

Referring now to FIG. 9 a perspective illustration is shown at 46, similar to that shown in FIG. 7 and depicting a related variant of bi-polar surgical forceps integrating a wire form within each beam. The overall shape and configuration of the surgical forceps is repeated from FIGS. 1-8 with the elongated beams jointed at rear ends by the over molded rear plug 12 with some overmolded material to secure the two tweezer tongs, and so that contact pins 14 and 16 extend therefrom. The beams each further again include an elongated body having rear, intermediate and forward extending interconnecting portions which establish a generally non-linear profile for maintaining enhanced viewing in use.

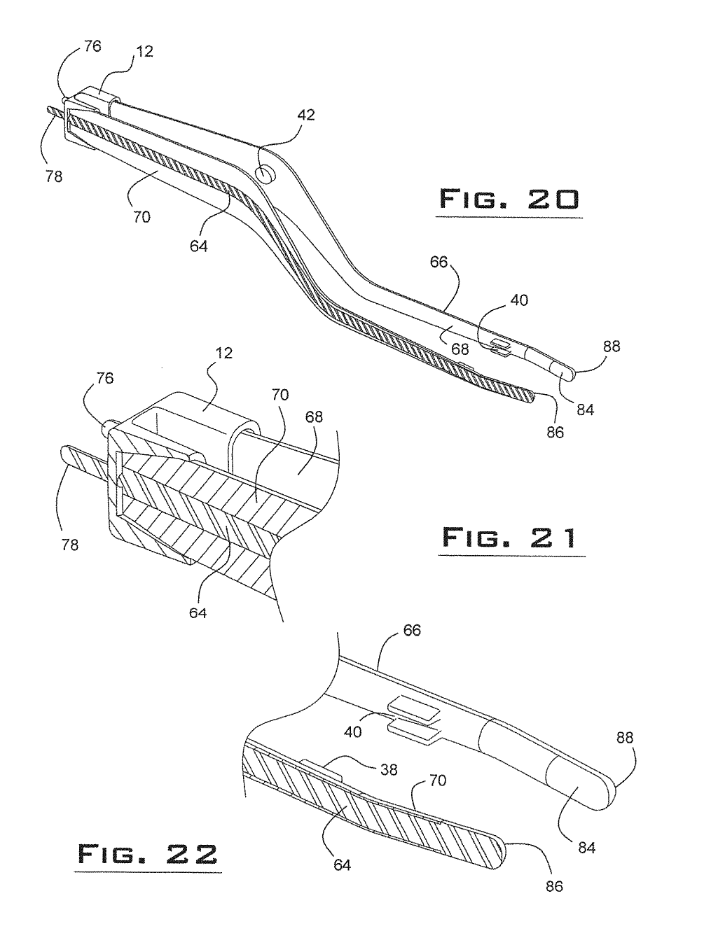

As best depicted in FIG. 12, a pair of elongated conducting wire forms are depicted at 48 and 50 in exploded fashion relative to the contoured beams. The wire forms 48/50 as shown provide the conductivity between the rear pin ends 14/16 and the forward probe tip ends 18/20 as substantially depicted in FIGS. 18 and are configured similar to the plastic over mold of the beams.

Formation of the beams according to this embodiment can include a first injection molding step in which half of the over mold material is coated about each of the conducting wire forms 48/50, following which a second injection molding step occurs in order to completely seal the wire forms in the manner shown so that their tips 18/20 project forwardly from the ends (at 52/54) of the forward beam portions 30/32. As further shown, the inner facing end surfaces of the wire form tips 18/20 may be further flattened or notched (see at 56/58) in contrast to the pointed profiles of the tips depicted in FIG. 7.

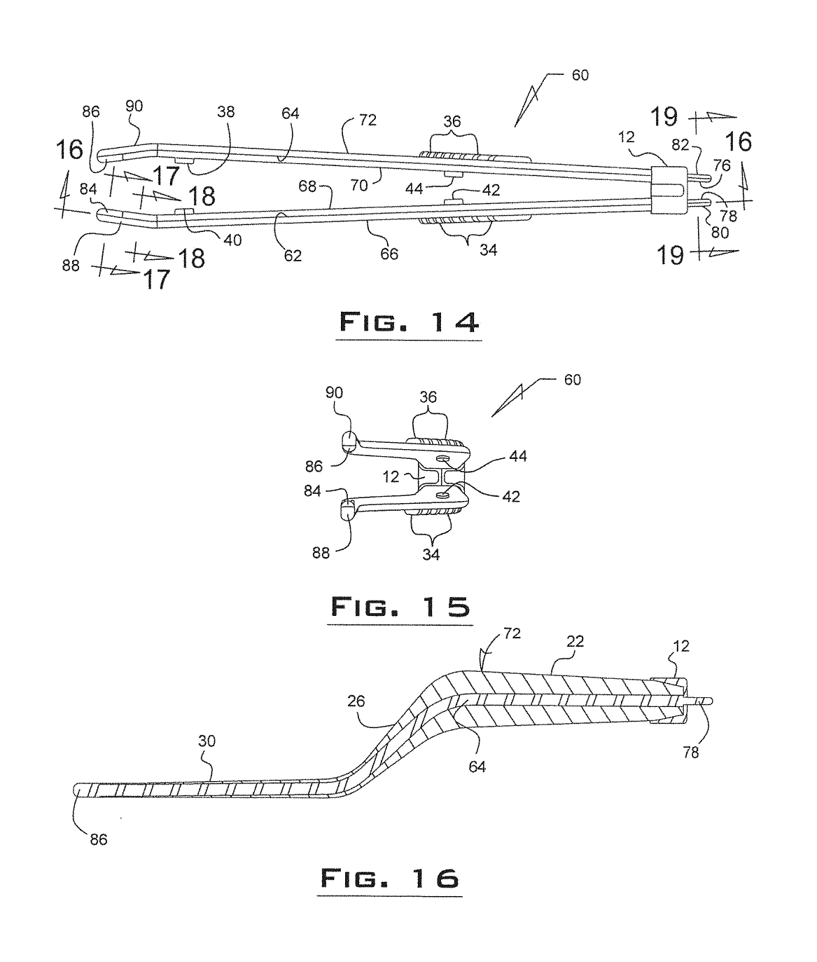

With reference now to FIGS. 14-23, a further related variant, generally at 60, is depicted of an electro-cauterizing surgical forceps in which the electrically conducting interior of the beams is reconfigured as a pair of metal platings or layers (at 62 and 64), such as gold or silver plated, and which can also include being formed of copper. As will be further structurally described, the metal layers 62/64 are formed into a pre-molded half portion of a selected beam (see as described in FIG. 23) and can be painted or coated with a heat/electrical insulating material, then assembled into the end housing 12 with some adhesive material to secure the two tweezer tongs to create the bipolar forceps.

The design of FIGS. 14-23 which enables the plastic to be plated with the conducting material (and as opposed to providing a separate wire form or stamping) provides a cost reduction with less materials and processes to manufacture. Existing forming processes include, in one assembly variant, providing the beams as individual pairs of halves (further reference being to joined halves 66/68 and 70/72 in FIG. 23) with one of the two plastic halves of each tong or beam being laser etched on the surfaces where gold plating (or silver or copper), will be applied. Formation can further include an injection molded composite that has a metallic additive, and when areas defined are laser etched, the plastic is burned off, exposing the metallic additive providing a metallic surface to bonding the plated metal. This process is understood to constitute available technology used for electrical circuit boards and circuit printed plastic components on surfaces with a profile/contour such that further explanation is not necessary.

The two plastic halves will then be either sonic welded together, or the non-plated half can be over molded onto the plated half to generate the tong assembly with the metal conduit sandwiched inside. The end housing will then be over molded with the two tweezer tongs to create the bipolar assembly.

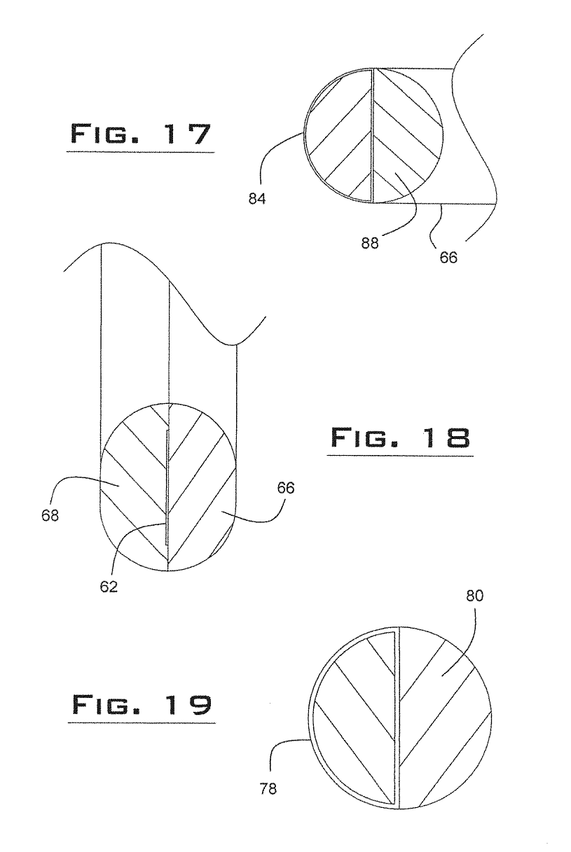

Proceeding from the above, and with initial reference to exploded FIG. 23, the plated portions are again depicted at 62 and 64, these being generally similar in configuration to the wire forms 48/50 of FIG. 12. The metal plated portions grated between split halves (see again pairs 66/68 and 70/72) of the outer plastic body. For purposes of ease of illustration, the beams formed by the split halves are similar in shape to that described in the preceding embodiments so that a repetition of the interconnecting rear, intermediate and forward end portions is unnecessary and beyond depicting such interconnecting portions 22, 26 and 30 associated with selected cutaway beam depicted generally at 72 in FIG. 16.

The bodies may be injection molded and subsequently laser etched (see at 74 for inside face of outer plastic half 70) in order to remove plastic material according to a desired depth and profile and to expose the metal additive which is then plated with the desired metal to provide the electrical conduit extending through each beam.

In this fashion, the metal platings 62/64 are applied to only one of the plastic halves (at 68 and 70 in FIG. 23) associated with each joining pair of halves 66/68 and 70/72. The second opposing halves 66 and 72 can be then heat welded or sonically welded to the exposed first halves. Alternatively, the second halves 66/72 can be over-molded to create the beam assembly for each side, following the laser etching process and so that the reconfigured rear half pins (inner opposing at 76/78) of each stamping 62/64 are paired with outer mating plastic pin extending portions 80/82 associated with the outer plastic halves 66/72.

As further shown, the forward extending ends of the stampings 62/64 further include expanded forward tip or probe ends 84/86 which define the contact pincer points for closing the circuit to apply current. Upon joining to the outer body halves 66/72 (again by heat/sonic welding or other molding techniques) the stamping tips 84/86 define the inwardly and opposingly facing surfaces of the probe tips, with forwardly most projecting plastic tip ends 88/90 of the outer plastic halves or shells 66/72 mating to the forward stamped tips 84/86.

With reference to the above, FIG. 14 is a top elongated view of the metal plated variant of bi-polar surgical forceps and illustrating the metal plated conduits 62/64 which extend between the projecting front probe ends 84/86 and rear pin ends 76/78, the interior running length of the assembled beams. FIG. 15 is a front end view of the surgical forceps of FIG, 14 and again illustrating the offset nature of the extending beams for providing enhanced and non-obstructed viewing by the surgeon during in situ surgical application, with FIG. 16 providing a ninety degree rotated angle of the forceps taken along cutaway line 16-16 in FIG. 14, and illustrating the metal plated interior 64 of the indicated beam, such omitting the need for metal stamping or wire forms.

FIG. 17 is again a crosswise cutaway taken along line 17-17 of FIG. 14 and depicting selected probe end 84 of selected beam 66 which includes the expanded inner facing contact portion associated with each conductive metal layer or plating. FIG. 18 is a further crosswise cutaway taken along line 18-18 of FIG. 14 and depicting the thin inner profile (see at 62) of the metal plating extending rearwardly from the forward probe tip ends through the interior of each pair of welded or otherwise joined beam halves.

FIG. 19 is a yet further cutaway taken along line 19-19 of FIG. 14 and depicting selected rear (inwardly facing) contact pin end 78 extending past the rear plastic over mold housing, the pin including metal plated inside facing half bonded to a plasticized outer half (see again at 80 which extends from a rearmost location of the outer beam plastic half 66 and which bonds to the metal contact half 78. As further shown in comparison to the forward probe end cutaway of FIG. 17 and the rear contact pin end cutaway of FIG. 19, the intermediate extending cross sectional profile of the thin layered or plated metal 62, is provided at a much thinner dimensional which provides material and cost savings while providing for adequate current conduction through the interior of the mating pairs of plastic beams.

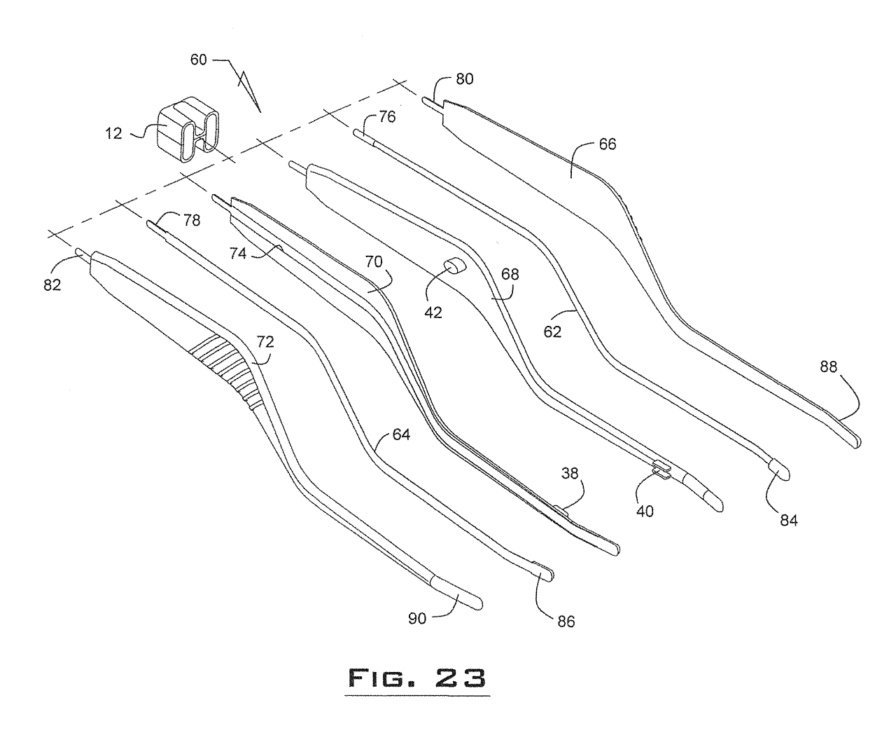

FIG. 20 is a rotated perspective view of the surgical forceps of FIG. 14 with selected outer facing beam depicted in partial lengthwise cutaway (outer half 72 depicted in FIG. 23 being removed) in order to reveal the interior metal plated portion 64 which is laser etched or otherwise, integrated into an over molded or sonically joined beam. FIG. 21 is an enlarged partial rear end perspective of FIG. 20, with the outside half facing plastic material again removed for better illustrating the inside metal plated surface of the selected contact pin 78.

FIG. 22 is an enlarged partial front end perspective of FIG. 20 which likewise depicts the outside plastic material (outer layer 72) removed for better illustrating the inside metal plated probe/tip surfaces (at 86) projecting from the forward end of the thin cross sectional plating 64. As best shown in FIG. 22, the interior height dimension of the thin plating 64 is reduced from the dimension of the probe tip 86 and so that the plating is recessed into the overlaying outer edge of the plastic mold half 70, such prior to the heat or sonic welding of the outer plastic half 72 as previously described.

By this construction, the gold or otherwise plated portions 62/64, such as applied to the plastic inner halves 68/70 can provide adequate electrical conduction through the beam interiors from rear pin end to forward tip end and upon the forward tip ends 84/86 of the platings 62/64 being contacted in pincer fashion in order to close a circuit and permit current to flow from the electrical source communicated with the remote rear pin ends 76/78 of the platings 62/64.

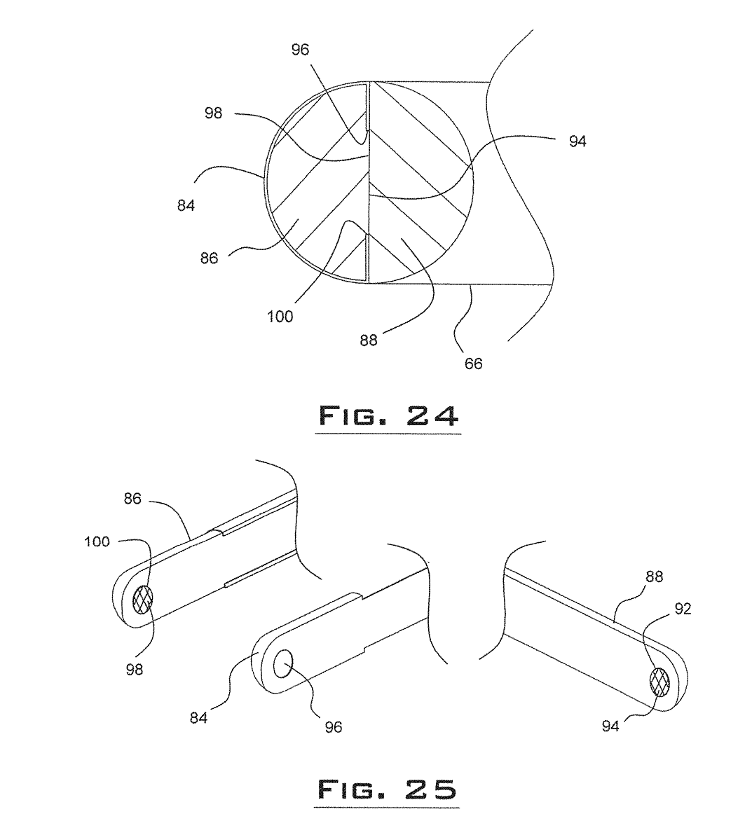

FIG. 24 is a cutaway of a probe tip end similar to that shown in FIG. 17 and illustrating an alternate variant for accomplishing secure attachment of a metal layer (e.g. gold or other metal plating at 84) at each inwardly facing probe end and which provides for a more secure attachment between the two plastic pieces a the tip with the gold plating for both tongs. FIG. 25 further illustrates a partial forward end perspective of a pair of inwardly facing probe ends as shown in FIG. 24.

As shown in FIG. 25, a surface area of any shape is provided on an inside surface of the forward end of the selected extending end portion of a beam (see at 92 for beam forward end portion 88 as well as at 100 for other beam forward end portion 86). In each instance, the shape will bond to a surface extending within the surface area (see as further designated at 94 for beam 88 and at 98 for beams 86) via sonic or heat welding to the respective opposing surface areas.

As further depicted in FIG. 25, a recess hole (see at 96) is formed in an exterior facing plastic side opposite the inwardly facing gold plated tip 84 (such as being of any matching shape) and which allows plastic material to extend from surface 98 thru aperture 96 to surface area 94. Further again shown at 100 is a round (or any other shaped) features which extends through the hole 96 in order to bond with the surface area 94. As is further understood, this bonding feature can be provided in any shape and can extend to the tip edges at 86 and 88 with a matching opening defined in the gold plating 84.

Having described our invention, other and additional preferred embodiments will become apparent to those skilled in the art to which it pertains, and without deviating from he scope of the appended claims.

We claim:

1. A bipolar forceps for providing electro-cauterization of tissue and blood vessels, comprising:
first and second elongated beams extending from a rearmost interconnected end to forward most separated probe ends, said beams each including rearmost base portions, intermediate angled portions and forward most extending portions such that said probe ends are established in a non-linear arrangement relative to said rearmost portions; and
first and second pins extending from said rear end and electrically communicating with said forward probe ends;

2. The forceps as described in claim 1, further comprising a plastic material over-molding a substantial length of each of said beams short of said probe ends.

3. The forceps as described in claim 2, further comprising a second over-molded plastic plug encapsulating said rearmost base portions of said beams and from which extends said pins.

4. The forceps as described in claim 2, further comprising pluralities of user gripping embossments arranged upon exterior opposite facing surfaces of at least one of said rearmost or intermediate angled portions of each of said beams.

5. The forceps as described in claim 1, further comprising interlocking features extending from inner opposing surfaces of said beams for preventing scissoring of said beams during pincering together of said forward ends.

6. A bi-polar forceps for providing electro-cauterization of tissue and blood vessels, comprising:
first and second elongated beams extending from a rearmost interconnected ends to forward most separated probe ends;
an elongated wire integrated into each of said beams and including a contact pin extending from a rear end and a probe configured at a forward end; and
a current source adapted to being communicated to said contact pins and through said wires upon closing a circuit at said forward probe ends.

7. The forceps as described in claim 6, said beams each further comprising rearmost base portions, intermediate angled portions and forward most extending portions such that said probe ends are established in a non-linear arrangement relative to said rearmost portions.

8. The forceps as described in claim 6, further comprising a plastic material over-molding a substantial length of each of said beams short of said probe ends.

9. The forceps as described in claim 7, further comprising a second over-molded plastic plug encapsulating said rearmost base portions of said beams and from which extends said pins.

10. The forceps as described in claim 7, further comprising pluralities of user gripping embossments arranged upon exterior opposite facing surfaces of at least one of said rearmost or intermediate angled portions of each of said beams.

11. The forceps as described in claim 6, further comprising interlocking features extending from inner opposing surfaces of said beams for preventing scissoring of said beams during pincering together of said forward ends.

12. A bipolar forceps for providing electro-cauterization of tissue and blood vessels, comprising:
first and second elongated beams extending from a rearmost interconnected ends to forward most separated probe ends;
said beams each further including a pair of joined plastic halves;
an elongated metal plating being applied according to any of an etching or plating operation upon an inner facing and exposed surface of a selected plastic half associated with each pair of joined plastic halves;
upon joining said pairs of plastic halves, said elongated metal layers being integrated into each of said beams, said metal layers also including a contact pin extending from a rear end and a probe configured at a forward end; and
a current source adapted to being communicated to said contact pins and through said wires upon closing a circuit at said forward probe ends.

13. The forceps as described in claim 12, a further selected plastic half associated with each beam further comprising a plastic extending rear pin mating with said metal contact pin.

14. The forceps as described in claim 13, said further selected plastic halves associated with each beam further comprising a plastic forward extending portion mating with said metal layer probe configured forward end.

15. The forceps as described in claim 14, further comprising an intermediate portion of each of said elongated metal layers extending between said joined plastic halves having reduced thickness dimensions in comparison to each of said metal contact rear pin and forward probe end.

16. The forceps as described in claim 12, said beams each further comprising rearmost base portions, intermediate angled portions and forward most extending portions such that said probe ends are established in a non-linear arrangement relative to said rearmost portions.

17. The forceps as described in claim 12, further comprising a plastic material over-molding a substantial length of each of said beams short of said probe ends.

18. The forceps as described in claim 16, further comprising a second over-molded plastic plug encapsulating said rearmost base portions of said beams and from which extends said pins.

19. The forceps as described in claim 16, further comprising pluralities of user gripping embossments arranged upon exterior opposite facing surfaces of at least one of said rearmost or intermediate angled portions of each of said beams.

20. The forceps as described in claim 12, further comprising interlocking features extending from inner opposing surfaces of said beams for preventing scissoring of said beams during pincering together of said forward ends.
